(19) United States
(12) Patent Application Publication
(10) Pub. No.: US 2011/0160544 A1
(43) Pub. Date: Jun. 30, 2011

(54) SYSTEM AND METHOD FOR ANALYSIS OF MEDICAL DATA TO ENCOURAGE HEALTH CARE MANAGEMENT

(75) Inventor: Gary A. Hayter, Oakland, CA (US)

(73) Assignee: ABBOTT DIABETES CARE INC., Alameda, CA (US)

(21) Appl. No.: 12/980,658

(22) Filed: Dec. 29, 2010

Related U.S. Application Data
(60) Provisional application No. 61/290,841, filed on Dec. 29, 2009.

(51) Int. Cl. A61B 5/00

(52) U.S. Cl. 600/300

(57) ABSTRACT

System and method analyze medical data of a patient having a disease afflicted health condition and action is taken to encourage the patient to perform wellness-enhancing activities and to take and report medical data more frequently. Consideration in the form of reduced insurance costs, medical supply costs, and medical equipment costs are given to patients who comply. Health care providers are advised that analysis of the patient's medical data is reimbursable and are also encouraged to perform such analyses through rewards. Patient data may be stored in a remote memory site, accessed by HCPs for analysis, and proprietary software may be used to communicate directly to insurance companies or other medical benefit entities.

### CROSS-REFERENCES TO RELATED APPLICATIONS

This application claims the benefit of U.S. application Ser. No. 61/290,841, filed Dec. 29, 2009, incorporated herein by reference in its entirety.

### BACKGROUND

The present invention relates to the collection, communication, and analysis of medical data, and, more particularly, to a system and associated method that analyze collected medical data to determine if management of a health care condition is occurring or is lacking

Diabetes mellitus, or simply, “diabetes,” is an incurable chronic disease. Type 1 diabetics must manage their diabetes by taking insulin to compensate for the rise in blood glucose that follows food consumption. Type 1 diabetes management works to prevent hyperglycemia, or high blood glucose, while especially averting the consequences of hypoglycemia, or low blood glucose, from over-aggressive or incorrect insulin dosing. Poor diabetes management can manifest in acute symptoms, such as loss of consciousness, or through chronic conditions, including cardiovascular disease, retinopathy, neuropathy, and nephropathy. Effective diabetes management requires effort.

Many different ways exist to assist in monitoring and managing one's glucose levels. Health care maintenance systems based on the use of a hand held device are often used. These devices are configured to record patient data such as blood glucose data. Additionally, it is known that such data can be uploaded to a remote server for storage of large quantities of medical data and later access to it by third parties, such as health care providers (HCP). Examples are Google Health and Microsoft HealthVault™. At the remote server location or elsewhere, blood glucose test results can be matched with quantitative information on medication, meals, or other factors, such as exercise.

Medical sensors can generate large quantities of useful information about a physiological parameter or parameters of a patient. That information, when processed, organized, and analyzed in particular ways, can be highly beneficial to a health care provider in examining the patient and recommending treatment. The appropriate calculations, organization, and analyses of that data can assist in forming rapid, useful, and more accurate evaluations of the information, the patient's history, and the patient's present state and health condition.

For example, analyte monitoring and medication delivery devices are commonly used in the treatment of a patient. One or more samples or analytes from the patient's body tissues is sensed and data is accumulated. A monitor, containing a sensor and a processor, may be used to acquire, accumulate, and process that data. Ultimately a report or reports must be produced from that data and an analysis made by a health care provider (HCP). In response to the analysis, one or more medications may be administered to the patient or other course of treatment prescribed, such as exercise and control over the timing, amount, and contents of meals. Administration of the medication may be manual by the patient such as self-injection with a syringe, by another person such as a nurse, or by a powered medication administration device, such as an infusion pump, for automatic or continuous delivery. For example, glucose monitors and insulin pumps are commonly used in the treatment and management of type 1 diabetes mellitus.

In the case of diabetes, a blood glucose monitor (BGM) or continuous glucose monitor (CGM) may be used in obtaining data about the glucose level of a patient. Such sensors detect glucose levels through actual analysis of a drop of blood, or through sensing the composition of interstitial tissue. The patient may have a hand held digital device, such as a personal digital assistant (PDA) that is used to receive and store his or her glucose data. This can occur in a number of ways. In the case where the patient draws a drop of blood onto a test strip that is read by a BGM, the data from the BGM may be communicated to the PDA for storage, processing (such as by adding a date and time stamp), and transfer elsewhere. In one case, the BGM is integrated with the PDA (dedicated device). In another case, the glucose data is communicated to the PDA wirelessly or through wired connection. In both cases of the BGM and CGM, various schemes may be used to get measured patient glucose data onto the PDA. The PDA is programmed to process that data and can provide a useful number representation of a glucose level on the screen of the PDA, and can also be instructed to upload the data to a server that may be remote and which may be accessed through the Internet (cloud computing) or by other means. Conveniently, a computerized report can be used to display such measurements and calculations of the measured glucose together and can be analyzed for use in developing health management recommendations. For example, glucose monitors are programmed to provide recommendations for better blood glucose management in the patient. Such analyses often include trends, extrapolations, predictions, alerts, and others.

The detection of the level of analytes, such as glucose, lactate, oxygen, and the like, in certain individuals is vitally important to their health. Moreover, recording analytics relating thereto, as well as other patient behavior such as activities and meals, and providing this information to health care providers for analysis can provide valuable, life-saving feedback to patients having difficult medical conditions. For example, the monitoring of glucose is particularly important to individuals with diabetes. Diabetics may need to monitor glucose levels, as well as diet and exercise, to determine when insulin is needed to reduce glucose levels in their bodies or when additional glucose is needed to raise the level of glucose in their bodies. Provision of related analytics to a health care provider may result in a therapy recommendation that may be useful in helping the patient better manage his or her diabetes. Existing data management and analysis tools are available and are further being developed to assist patients along these lines.

However, one obstacle is that the analysis may be time consuming and difficult for some doctors. Further, many doctors are also unaware that reimbursement is available from health care companies, Medicare, etc., for such analysis. Moreover, a lack of health care provider interaction in the monitoring process has the potential to drastically undercut the benefits available in existing disease, diabetes, and analyte management regimes. As a result, there is a need for systems and methods that inform health care providers, facilitate their involvement, and/or otherwise encourage feedback, interaction, and reward in such management regimes.

The health insurance industry, companies that pay health insurance premiums, and others that are responsible for health care have recently started to encourage people to improve their health by providing direct health and wellness coaching. They reward people for taking standard health tests, discussing the results with a health advisor and engaging in wellness-enhancing activities. Often the reward is a discount on the employee portion of the health insurance premiums or discounts on other goods and services.

However, one obstacle to participation in such programs is that obtaining the requisite feedback from a health care provider may be too time consuming and/or difficult for many health care recipients to perform. Further, many patients and doctors are also unaware that rewards, reimbursement or other consideration may available for the provider's time in preparing such analyses. Such disincentives to participation in wellness-enhancing activities may reduce the efficacy of a patient's diabetes management plan.

Hence, those of skill in the art have recognized that there is a need for systems and methods that incentivize and facilitate greater participation in wellness-enhancing programs and improvements in disease management. Those skilled in the art have also recognized a need for encouragement to patients to collect and communicate health condition data for analysis by a skill health care professional. Another identified need is the analysis of collected patient data by a health care provider or professional. A further need is to notify analysts of the opportunity for reimbursement for performing an analysis of patient data. The present invention fulfills these needs and others.

### SUMMARY OF THE INVENTION

Briefly and in general terms, the present invention is directed to a medical data management system and method that encourage more rigorous management of a disease afflicted health condition.

Systems, methods and computer-readable media are adapted to encourage and enable a person to perform a wellness-enhancing activity and report such performance. The invention provides processing instructions whereby the person is rewarded for one or both of performing the wellness-enhancing activity and/or achieving health data of an improved value, quantity, quality, or profile, or whereby the person is cautioned to improve their health management regimen.

In one aspect, there is provided a method of processing instructions associated with enabling a patient to report/perform a wellness-enhancing activity and processing instructions regarding rewarding the patient for one or both of performing the wellness-enhancing activity and/or achieving health data of an improved value, quantity, quality or profile.

More particularly, the method may include one or more of the following steps: (1) processing instructions associated with enabling the patient to perform a wellness-enhancing activity, wherein the wellness-enhancing activity may include compliance with a health-related data reporting protocol, improved management of a disease condition, or engaging in health-promoting activities such as exercise or eating more nutritious foods; (2) reporting the performance of the wellness-enhancing activity to a health care provider (analyst) at particular intervals; (3) analysis of the reported data by the analyst; (4) consultation between the analyst and the patient; (5) submission of information relating to any of the prior steps to a database supported by the analyst or a third party, such as a health insurer; (6) providing the individual and/or analyst with an incentive to engage in further wellness-enhancing activities and/or a reward for doing so, which incentives and rewards could include, without limitation, discounts on health care-related products, reimbursement to the analyst for time spent in performing the methods of the invention, and other appropriate consideration to improve management of a disease condition and/or general wellness.

In accordance with a system, there is provided a medical data analysis system to encourage more rigorous management of a health condition, the system comprising a medical sensor configured to sense a physiological parameter relevant to a particular disease-afflicted health condition and to provide medical data representative of the sensed parameter, a processor programmed to receive the medical data that is representative of the sensed parameter and relevant to the afflicted health condition, process the received medical data, process an analysis of the afflicted health condition that is based on the medical data, and automatically determine the status of the afflicted health condition based on the analysis, and the processor further programmed to automatically provide a level of encouragement to more rigorously manage the afflicted health condition, wherein the level of encouragement is based on the determined status of the afflicted health condition.

In more detailed aspects, the encouragement comprises a reward and wherein the processor is further programmed to automatically provide the level of reward based on the determined status of the afflicted health condition. The reward comprises an automatically granted discount on a health insurance cost. The reward comprises an automatically granted discount on co-payment for office visits. The encouragement comprises a penalty and wherein the processor is further programmed to provide the level of penalty based on the determined status of the afflicted health condition. The encouragement comprises a reward and wherein the processor is further programmed to a provide a reward for the more frequent sensing of a physiological parameter relevant to the particular disease-afflicted health condition and for more frequently providing medical data representative of the sensed parameter to the processor for processing.

In yet other system aspects, the processor is further programmed to provide a report of the processed medical data, the analysis, and the determined status of the afflicted health condition. The system further comprises a memory located remotely from the processor, and wherein the processor comprises a plurality of processors, one of which is programmed to receive the medical data that is representative of the sensed parameter and relevant to the afflicted health condition, process the received medical data, and store the processed medical data in the memory. Another of the plurality of processors is programmed to retrieve the stored medical data from the memory, process an analysis of the afflicted health condition that is based on the medical data, automatically determine the status of the afflicted health condition based on the analysis; and automatically provide a level of encouragement to more rigorously manage the afflicted health condition, wherein the level of encouragement is based on the determined status of the afflicted health condition. Yet another of the plurality of processors is programmed to automatically determine the status of the afflicted health condition based on the analysis; and automatically provide a level of encouragement comprising a reward when the determined status indicates that the afflicted health condition has improved.

In other detailed system aspects, the reward comprises at least one of an automatically granted: discount on a health insurance premium, free medical service, discounted medical service, discounted medical supplies, discounted medical equipment, coupon redeemable for medical supplies, recognition, and congratulations. The processor is further programmed to automatically vary the provided level of encouragement as a function of a change in diet. The processor is further programmed to automatically vary the provided level of encouragement as a function of the frequency of sensing the physiological parameter relevant to the particular disease-afflicted health condition and providing the sensed medical data to the processor.

Further aspects include that the processor is further programmed to store the medical data in a memory that is accessible by a health care provider and to notify the health care provider that analysis of the stored medical data is reimbursable. In further detailed aspects, there is provided a medical data analysis system in which the medical data is related to diabetes, the system to encourage more rigorous management of diabetes, the system comprises a glucose sensor configured to sense a glucose level and to provide glucose level signals, a processor programmed to receive the glucose level signals, process the received glucose level signals, process an analysis of the diabetes based on the glucose level signals, and automatically determine the status of the diabetes based on the analysis, wherein the analysis of the diabetes based on the glucose level signals is prepared by a health care provider, the processor further programmed to automatically provide a level of consideration to more rigorously manage the diabetes based on the determined status of the diabetes, with consideration automatically provided as a function of at least one of: an improvement in diet, performing a wellness-enhancing activity, an improvement in glucose level measurements, and wherein the consideration comprises at least one of: a discount on a health insurance premium, a free medical service, a discounted medical service, discounted medical supplies, discounted medical equipment, a coupon redeemable for medical supplies, recognition, and congratulations.

In yet more detailed method aspects, there is provided a method of encouraging improved management of a person's health in which the method comprises sensing a physiological parameter relevant to a particular disease-afflicted health condition and providing medical data representative of the sensed parameter, receiving and processing information regarding performance of a wellness-enhancing activity, processing the medical data and the information regarding the wellness-enhancing activity, analyzing the medical data representative of the sensed parameter and the information regarding the wellness-enhancing activity and providing an analysis of the status of the afflicted health condition, and automatically providing a level of encouragement to more rigorously manage the afflicted health condition based on the analysis.

More detailed method aspects include the step of automatically providing a level of encouragement comprising automatically rewarding by providing at least one of: a discount on health insurance cost, free or discounted services, discounts on supplies, discounts on equipment, recognition, and congratulations. The step of automatically providing a level of encouragement comprises automatically rewarding for at least one of: an increase in frequency of measuring health-related parameters for obtaining the health data, an improvement in diet, and an improvement in the health data. The analysis of the status of the afflicted health condition is further automatically provided to an insurance provider or insurance billing entity to change the insurance policy cost.

Various features and advantages of the invention will become more apparent by the following detailed description of several embodiments thereof with reference to the attached drawings, of which:

### BRIEF DESCRIPTION OF THE DRAWINGS

FIG. 1 is a block diagram of an exemplary health care system including a patient or “user” of the system, a health care provider environment, and a health care insurance entity, showing a data network interconnecting all entities;

FIG. 2 is a block diagram of an exemplary health care provider environment consistent with FIG. 1 and showing further details of aspects of embodiments of that environment with certain programming being shown;

FIG. 3 is a block diagram similar to that of FIG. 1 showing an exemplary health care system with a patient, health care provider environment, and insurance entity, and also including a further provider related to the health care of patients/users;

FIG. 4 is a block diagram of an embodiment of a health care system in accordance with aspects of the invention showing the use of certain programming to facilitate the management of health care of a patient or individual, and including links to an insurance entity and an employer;

FIG. 5 is a flow chart of a method illustrating the use of analysis of health measurement data in creating encouragement for patients to manage health conditions;

FIG. 6 is similar to FIG. 5 in that it presents a flow chart of a method illustrating the use of analysis of health measurement data in creating encouragement for patients to manage health conditions but in addition, it further includes the step of operating a health care improvement program;

FIG. 7 also presents method steps in analyzing medical data to determine the existence of improvement in managing a health condition and determining encouragement;

FIG. 8 shows a method of processing different sets of medical data for an analysis to determine that a health improvement action was taken and its result, as well as the use of that analysis in encouraging the patient.

### DETAILED DESCRIPTION OF THE PREFERRED EMBODIMENTS

Reference will now be made in more detail to the drawings, wherein like reference numerals refer to like elements throughout. The invention is directed to a system and associated method to encourage and enable a patient to perform wellness-enhancing activities and have them reported in a fashion that makes the patient eligible to receive wellness incentives and rewards. In one embodiment, there is provided a method of encouraging individuals and/or their health care providers to engage in more rigorous management of a health condition and to engage in behaviors likely to better maintain the person's health. To that end, a method includes one or more of the following steps: (1) processing instructions associated with enabling the patient to perform a wellness-enhancing activity, wherein the wellness-enhancing activity may include compliance with a health-related data reporting protocol, improved management of a disease condition, or engaging in health-promoting activities such as exercise or eating more nutritious foods; (2) reporting the performance of the wellness-enhancing activity to a health care provider (analyst), insurer or wellness coach at particular intervals (collectively, an “analyst”); (3) analysis of the reported data by the analyst; (4) consultation between the analyst and the patient; (5) submission of information relating to any of the prior steps to a database supported by the analyst or a third party, such as a health insurer; (6) providing the individual and/or analyst with an incentive to engage in further wellness-enhancing activities and/or a reward for doing so, which incentives and rewards could include, without limitation, discounts on health care-related products, reimbursement to the analyst for time spent in performing the methods of the invention, offers for free services, discounts on health insurance premiums and other appropriate consideration to improve management of a disease condition and/or general wellness. The patient and/or analyst can also be provided with reports regarding savings realized over a period of time as well as forecasts as to additional savings that could be realized with further improvements in wellness management. Details of such implementations as well as other exemplary implementations, are set forth in more detail further below.

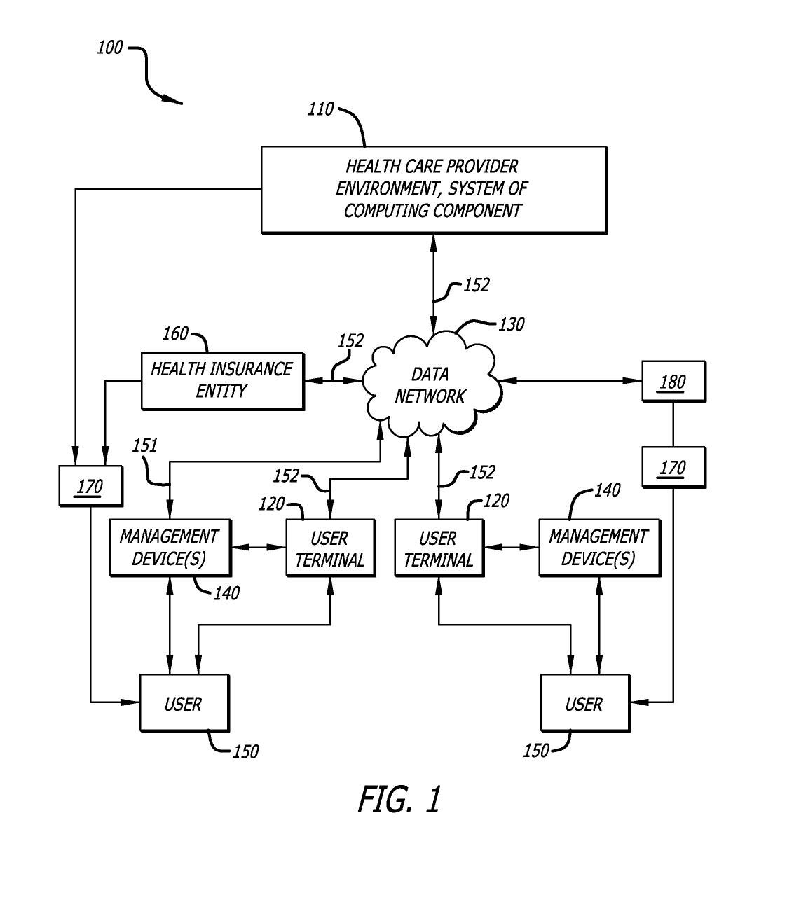

FIG. 1 illustrates a block diagram of a patient care and health care/insurance environment system 100 (“patient care system 100”) in accordance with one or more implementations of the innovations herein. The diagram 100 includes an environment, system, or computing component 110 that is associated with one or more health care providers (“HCP environment 110”) and is operatively coupled to one or more user terminals 120, health management devices 140, and other components via a data network 130. As can be seen from FIG. 1, each of the user terminals 120 are associated with patients 150 and may also be configured to be operatively connected to a respective one or more management devices 140 (e.g., testing, monitoring, etc. devices). As will be discussed in more detail below, there is also provided a health care management organization/insurance entity computing component/environment 160 (“health insurance entity 160”) operatively coupled to the data network 130 for communication with the analyst environment 110, user terminals 120, and other components associated with the network 130.

The patient care system 100 may also include an employment/benefits management-related entity 180 (“employer 180”) that is associated with one or more of the patients 150 and/or the health insurance entity 160, and may also be connected to the data network 130. The employer 180 is also, optionally, connected to the data network and/or software systems associated with or used by the patients 150 and/or health insurance entity 160. Further, the employer 180 is associated with discounts or other incentives and rewards that are provided by employer to a patient 150 consistent with aspects of the innovations set forth herein.

Rewarding the patient 150 may take the form of payment, credit, recognition or other consideration 170 (collectively, “consideration 170”) provided to a patient via a variety of means, mechanisms, and channels. According to one illustrative channel, the health insurance entity 160 may provide consideration 170 to the patients 150 consistent with aspects of the innovations herein. In such arrangements, the health insurance entity 160 (alone, or in connection with a health care provider) receives and analyzes medical data or related information regarding a patient's condition, treatment, prognosis, disease management history, and wellness-enhancing activities. For example, the health insurance entity 160 could monitor a patient's glucose measurements and/or other data and provide consideration or additional reward for improvements in the patient's health and/or disease management or for maintaining a favorable glucose profile. Similarly, the consideration/reward could be reduced for repeated material non-compliance with a prescribed course of diabetes management, as indicated by data indicating such.

Here, as with other exemplary implementations, the functionality may be enabled via software modules and/or one or more websites that provide capabilities of uploading data, transmitting, reviewing, and/or analyzing information or results, and providing various features, such as purchasing options, notifications to the patient, adjustments to insurance premiums, and/or offering discounts including, e.g., coupons for third-party purchase discounts. Various functionality and analyses may also (or, instead) be made performed via one or more computing components or devices associated with the particular health care regime. In particular, interaction of various remote computing components with the website provides advantageous functionality consistent with the innovations herein. For example, provision of analysis and feedback to a patient via a CGM (continuous glucose monitoring) device or remote, as set forth herein, enables a patient to provide (glucose, etc.) measurements and immediately receive a reward/consideration, thereby encouraging exchange of information and performance of wellness-enhancing activities.

As explained in part above, the health insurance entity 160 may receive health care measurements or information regarding a patient 150 from entities and/or computing components within the system 100, such as the health care provider environment 110. In other words, while the health care information may relate to specific patient data, it may be obtained or sent from any of computing component within the health care regime. For example, the health care provider environment 110 may serve as the repository and analysis site for the patient health care measurements.

Here, information and instructions may be generated via health care provider software resident in the health care provider environment 110 and sent to the health insurance entity 160. These information and instructions may take the form of simple instructions for the health insurance entity to reimburse a patient with various forms of consideration 170 set forth herein, such as providing reductions of insurance premiums, other monetary, credit or debit incentives, etc. Furthermore, such exchange of information and instructions may take the form of interrelated software components that provide consideration or other insurance/reimbursement functionality in the context of automated systems or subsystems that assist the health care providers with analysis of patient data and even recommendations for treatments or responses thereto. For example, the health insurance entity may be in partnership with, and/or utilize a common website or software platform provided by, an entity 160 that supplies analyst software in the health care provider environment 110. Further, a common website or software platform may provide space for educational or advertising messaging, such as geographically-tailored promotions for participation in local wellness activities (e.g., fitness programs).

In some implementations, the management device 140 may include one or more patient monitoring devices such a glucose meter (e.g., a glucose meter that includes wireless communication capabilities) which is configured to automatically and wirelessly transmit the measured glucose data to the network 130 or other relevant computing devices at a predetermined frequency via a wireless connection 151 and/or network 130. Here, for example, patient information such as glucose data may be transmitted to the network, to other website components or portals, to the analyst environment 110, or to other relevant entities set forth herein. In situations where the analyst environment 110 is the recipient of and/or performs processing of patient health measurements, a computing component within the analyst environment 110 may be configured to obtain or receive patient data, such as measured glucose data, from the testing or monitoring (management) devices 140 and store the received data in a corresponding user account associated with the patient 150 or the management devices 140.

It can be seen that each of the user terminals 120, the health insurance entity 160, and the analyst environment 110 are operatively coupled to the data network 130 via links such as data communication links 152. These data communication links 152 may take various forms, and may include wired or wireless communication paths which may be configured for secure, encrypted bi-directional data exchange over the data network 130. In particular, the data communication link 152 in one embodiment may include one or more of Wi-Fi data communication, IrDA data communication, infrared data communication, Bluetooth data communication, ZigBee data communication, USB or Firewire cable based data communication, Ethernet cable based data communication, and dial up modem data communication.

For example, in some implementations, the user terminals 120 may include one of a personal computer (including a desk top or a laptop computer) or a handheld communication device such as a Blackberry or other smart phone, Internet access enabled mobile telephones, a bi-directional communication enabled pager, and a communication-enabled personal digital assistant (PDA). In one implementation, the user terminals 120 include an output unit such as a display and/or speakers, an input unit such as a keyboard or a touch-sensitive screen, as well as a controller such as a CPU for performing user-instructed procedures at the user terminals 120. Moreover, within the scope of the present invention, the user terminals 120 may be configured to communicate with the data network 130 using a wireless data communication protocol such as Bluetooth, 801.11x, and ZigBee. Additionally, the user terminal 120 may be also configured to communicate with the testing or monitoring device 140 via short range RF communication path, an infrared or IrDA communication path, or using Bluetooth communication protocol. Additionally, the management (testing or monitoring) device 140 may also be configured to connect to the respective user terminals 120 via a wired connection such as a USB connection, an RS-232 cable connection, or an Ethernet cable connection.

The health insurance entity 160 may be configured to communicate with the analyst environment 110 and the user terminals 120 over the data network 130 using either a wired or a wireless secure and encrypted connection. As is generally the case, because the relevant patient and/or reimbursement information may be very sensitive, a high level of security for data communication to and from the health insurance entity 160 and other parties to such communications may be used such as encryption level exceeding 128-key encryption, and the like. Within the scope of the present invention, the health insurance entity 160 may be affiliated or have a consideration-sharing arrangement with a provider of services or supplies to analysts 110 or patients 150, a provider of software used by the analysts and/or patients, a banking institution terminal, a credit card institution terminal, a brokerage institution terminal, and any other financial institution terminal which maintains a financial account of a user and by which consideration may be commoditized to provide consideration 170.

Accordingly, individuals enrolled in various health insurance programs (referred to as “patients” herein for the sake of convenience) benefit from such systems and arrangements by generally encouraging better health and therefore reducing overall health costs. With regarding to providing glucose data, for example, people with diabetes would not only enjoy improved health but generally lower premiums as well as, optionally, other direct rewards/consideration. Again, such rewards/consideration may be discounted premiums, though may also include free or discounted services, discounts on supplies or equipment (e.g., strips, meters, etc.), and recognition, among the other mechanisms and means herein.

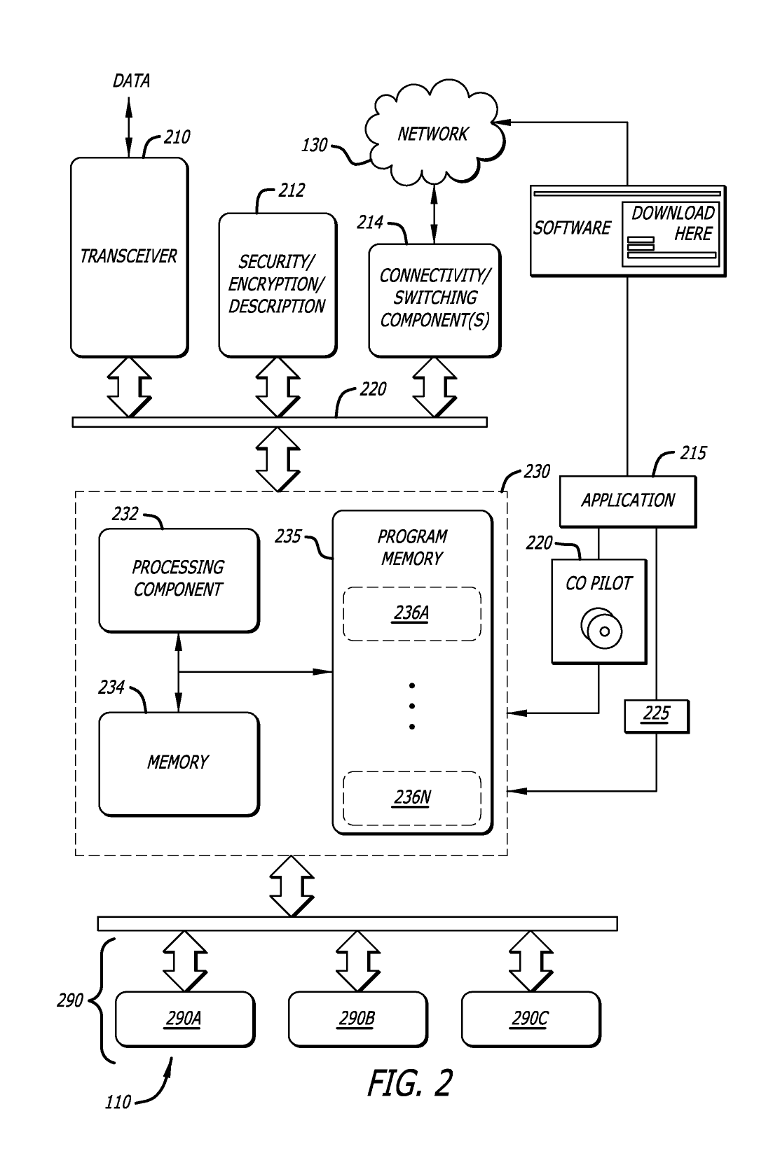

Referring yet again to FIG. 1, computing components associated with the health insurance entity 160, the analyst environment 110 and other relevant entities may, in some implementations, include one or more computing devices, such as one comprising a controller operatively coupled to an input/output (I/O) interface unit, a read-only memory (ROM), a random access memory (RAM), and data store(s). In general, in some implementations, such processing components and/or data store(s) may include or run a server application and an operating system. In this manner, as is set forth in more detail in connection with FIG. 2 below, such controllers may be configured to communicate with the user terminals 120 and the other entities in the system 100 over the data network 130 via an I/O interface unit, under the control of the various processes and routines stored in the ROM, the data store(s), etc., and/or associated with user transmitted requests and information.

According to further implementations, such server applications and operating systems of the data store(s) may be configured to provide a proprietary interface for the users, to execute secure and encrypted data communication over the data network 130. More specifically, one or more proprietary internet-based user interfaces may be provided at a predetermined URL for users to login using a variety of computing platforms. For example, special interfaces may be provided to the patients, the health care providers, the health insurance entities 160 and/or the employers 180, where these interfaces may be enabled via software resident in or managed by any one such component, or via software provided to one or more of these entities by an independent (third-party) provider. Alternatively or in addition, within the scope of the innovations herein, the data network 130 may include the Internet, wherein the server application(s) and the operating system(s) of the system 100 members are configured to provide a dedicated website for allowing the relevant users, such as patients, health care providers and the employment/insurance entities, to securely and easily login using terminals over the data network.

FIG. 2 illustrates an exemplary configuration of an exemplary computing device, component, or environment consistent with aspects related to the innovations herein. Referring to the exemplary system 200 of FIG. 2, an exemplary computing component 230 may be implemented as a device configured for communication with other computing components or platforms throughout the overall environment 100, and may include a processing component 232, at least one memory component 234 (e.g., RAM, etc.), and program memory 235 (e.g., ROM, Flash memory, disk or optical drives, CD-ROMs, etc.). Further, the analyst computing component 230 may have one or more of a variety of input/output devices 290, such as a keyboard, mouse, etc., a display, and/or auditory or other human/sensory interactive components.

The computing component 230 may include or be associated with a transceiving component 210 to enable communication via various channels and regimes, including transmission and/or receipt of information composed of or including MPEG-encoded digital video and audio data, digital data, software programs, data files, and the like. The analyst computing component 230 may also be associated with other data processing components 212 (e.g., security, encryption/decryption, etc.) and/or additional connectivity components 214 such as routing/switching components (e.g., back channel connection components, etc., such as network cards, routing components, RF receivers, ports, receivers, etc.) giving access to data networks such as network 130 to provide connection to components associated with such networks.

Such computing components 230 may have an underlying health care/insurance application, or module 215 (“health care application 215”), already installed, and/or the systems and methods herein may include providing such an application via computer readable media 220 such as one or more CD-ROMs, via other memory devices 225 including computer readable media, digital media, flash drives, optical drives, etc., or via electronic communication such as wired or wireless transmission, e-mail, download over a network 240 such as the Internet, etc. The computing component 230 may be configured to run an operating system which supports multiple applications. Such operating system is typically a multitasking operating system that enables simultaneous execution of multiple applications. In some implementations, the operating system may utilize a graphical user interface environment that implements the applications or documents in a window-type operating system, such as Microsoft Windows, OS2 by IBM, or Apple Macintosh. Once resident on the computing component 230, such health care application(s) 215 may be utilized as set forth herein, e.g., to facilitate provision of reward/consideration to patients in the contexts of providing health care information, performing wellness-enhancing activities, etc. as set forth herein.

For example, health care application or component 215 may be used to implement a computer-based data management system known as the CoPilot™ Health Management System (CoPilot™ system). The CoPilot™ system is a personal computer (PC or portable or handheld appliance)-based software application that permits people with diabetes, their health care team, and caregivers to upload data from FreeStyle™ and Precision Xtra™ blood glucose monitoring systems (and generally from several other commercially available blood glucose meters and insulin pumps) into the CoPilot™ system application.

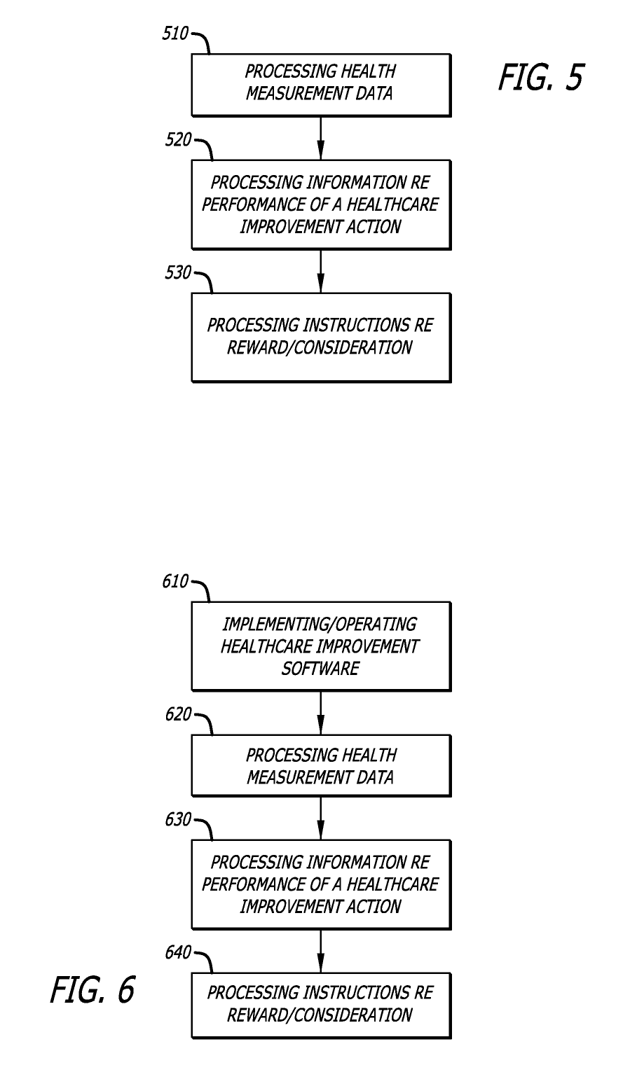

The CoPilot™ system provides an accessory to a blood glucose monitoring system such as the FreeStyle™ and Precision Xtra™ blood glucose monitoring systems and other commercially available blood glucose meters and insulin pumps. The application provides graphs and other software tools for people with diabetes and their health care professionals (analysts) to help evaluate and analyze medical information such as glucose readings, carbohydrate intake, insulin dosage, exercise and other diabetes-related factors uploaded from devices or manually entered into the system. The system can help identify trends that can be used to educate persons with diabetes to improve their glucose control, for example. In accordance with an aspect of the invention, and as described further hereinbelow with respect to FIGS. 5-8, such a software application can be configured to employ various means to provide analysts with reimbursement information or options.

Additional detailed description of the above-described PC-based software application for health care management and its various features and functionality are provided in U.S. patent application Ser. No. 11/146,897 filed Jun. 6, 2005, published as US2006/0010098A1, entitled “Diabetes Care Report generation Architecture and Data Management System,” assigned to the Assignee of the present application and incorporated herein by reference in its entirety.

Alternative applications for health care management of use in accordance with aspects of the invention are those employed by third parties, such as health insurance entities. For example, an insurer-maintained database for uploading patient data could be password-protected and accessible to the patient via the internet.

Referring to FIG. 2, the memory 234 and/or other data store(s) of the analyst computing component 230 in some implementations may be configured to store data and information related to the patients and/or patient accounts such as, but not limited to, identification, user contact information such as telephone and/or facsimile numbers, email address, billing and shipping addresses, user account profile information such as replenishment level information, seasonality or periodicity of user use of the testing or monitoring device, user financial account information (for example, a bank routing number and bank account number in the case of a banking institution), and user testing or monitoring device data information such as the user, test strip order history, health related monitoring data such as previously measured glucose levels, user specific basal profile information, bolus determination information, insulin sensitivity, trend information determined based on the measured glucose levels (e.g., determined by the controller, etc.), and additional health care provider information for the patient such as contact information for the patient's other physicians, hospitals, nursing facilities, etc.

The computing component 230 also typically includes the program memory 235, which may include a variety of program modules 236A-236N, such as communication components that enable sessions, authorize transmissions, etc. between the analyst computing component 230 and other elements of the overall environment 100. Examples of such modules may include, but are not limited to, software modules such as encryption/decryption, key handling, hashing, signing, authentication, CAPI, CSPs, etc., implemented via dynamic link libraries and/or other existing techniques.

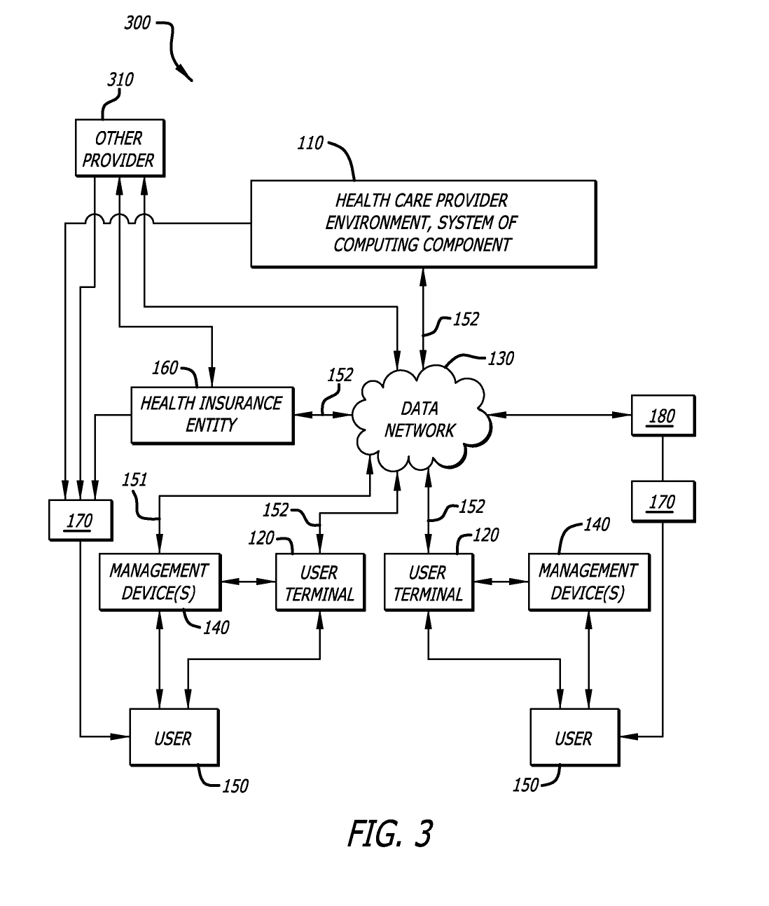

FIG. 3 is a block diagram of an exemplary patient care and health care provider environment 300 with health care/insurance features very similar to FIG. 1, illustrating further exemplary consideration channels consistent with the innovations herein. In addition to elements shown and described in FIG. 1, the exemplary environment 300 illustrates a variation of the innovations herein wherein an additional business, provider, or supplier (“other provider 310”) may be involved in the health insurance and/or reward/consideration 170 provision functionality. Other providers may be interconnected with the other entities directly or over the network 130, and may operate using the same software or platform (e.g., CoPilot™ system) as one or more of the other entities in the operating environment 300. In one exemplary implementation, here, other provider 310 may be a supplier business, e.g., a medical device supplier such as a business that provides, as the consideration 170, glucose test strips or meters, or discounts thereon. Further, the other provider 310 may take the form of other businesses or service providers, such as a health food product supplier, a health service supplier, etc., which may provide products, services, discounts thereon, or other rewards/consideration 170 related to their products or services.

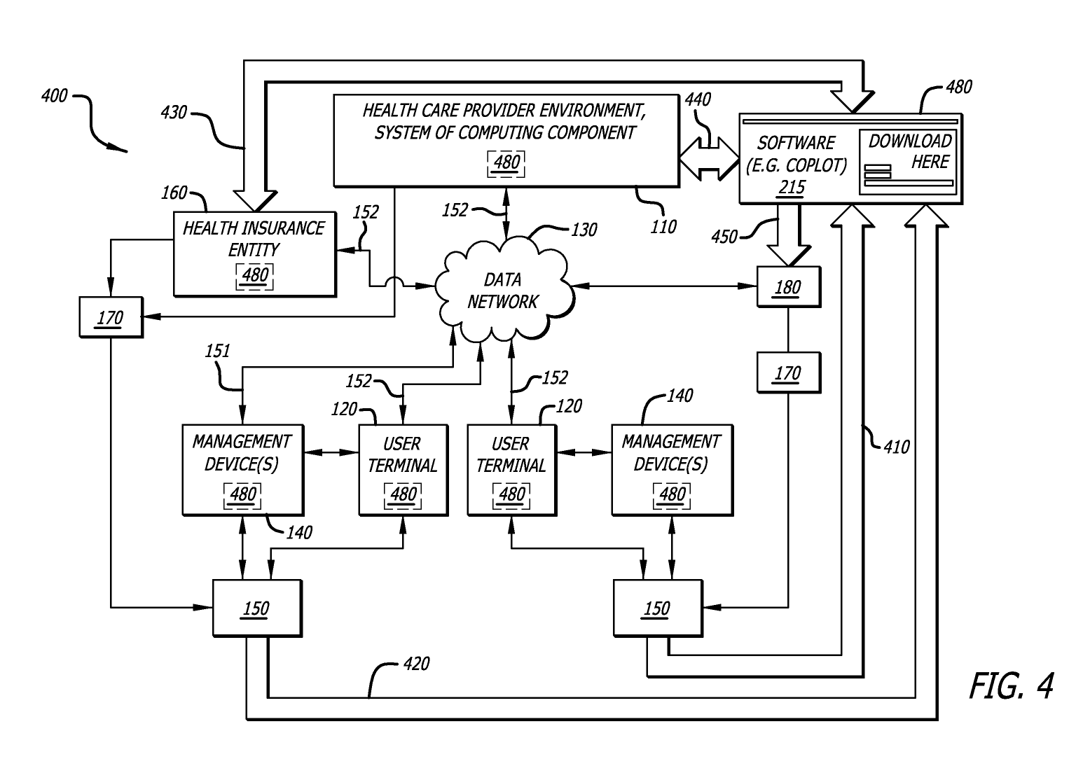

FIG. 4 is a block diagram of an exemplary patient care and health care provider environment with health care/insurance features consistent with certain aspects related to the innovations herein. Exemplary information processing consistent with one or more aspects of the innovations herein having a software platform 480 including a health care application 215 (e.g., CoPilot) is disclosed. As an initial step in some exemplary implementations, health measurement information 410, 420 may flow from the patients 150, from the user terminals 120, or from the management devices 140 and be transmitted to the network 130, i.e., for provision via the software platform 480, eventual transmission to the other computing components in the environment 400, etc. Such information may then be processed 440 by the health care provider 110 via the software platform 480, with adjustment information then being exchanged 430, 450 with the health insurance entity 160 and/or employer 180. In turn, these entities may then provide the reward/consideration 170, such as discounts to health insurance, etc., as discussed herein, to the patients 150.

FIG. 5 is a flow chart illustrating exemplary health care/insurance provider environment functionality consistent with certain aspects related to the innovations herein. As shown in FIG. 5, an exemplary method of encouraging management or improvement of patient health may comprise processing health measurement data 510 (e.g., data related to original information flow 410, 420 from or related to the health care recipients, etc.), processing information regarding performance of a health care improvement action or wellness-enhancing activity 520 (e.g., processing instructions regarding providing an improved health measurement, maintaining a favorable health measurement or profile, etc.), and processing instructions or information regarding reward/consideration 530 (e.g., processing instructions regarding any of the various reward and/or consideration features herein). For example, one specific implementation, here, may comprise processing patient glucose measurement data by the health care provider via a web portal 480 (FIG. 4), processing information to confirm that the patient has improved his/her glucose profile, and notifying the patient (e.g., via an email) that his/her health insurance premium is being reduced. Such notifications would preferably include links to relevant third party websites with wellness-related resources.

FIG. 6 is a flow chart illustrating further exemplary health care provider environment functionality consistent with certain aspects related to the innovations herein. As shown in FIG. 6, an exemplary method of encouraging management or improvement of patient health may comprise implementing or operating health care improvement software 610 (e.g., installing or operating Copilot™ system, etc.), processing health measurement data 620 (e.g., data related to original information flow 410, 420 from or related to the health care recipients, etc.), processing information regarding performance of a health care improvement action or wellness-enhancing activity 630 (e.g., processing instructions regarding providing an improved health measurement, maintaining a favorable health measurement or profile, etc.), and processing instructions or information regarding reward/consideration 640 (e.g., processing instructions regarding any of the various reward and/or consideration features herein). For example, one specific implementation, here, may comprise implementing a Copilot™ system web portal by which a patient may upload glucose data, processing patient glucose measurement data by the analyst via the web portal, processing information to confirm that the patient has improved their glucose profile, and providing the patient with a coupon for purchase discount(s) from a third-party entity.

Alternatively, the patient data may be uploaded to a third party website, such as the site of the patient's health insurance entity. In detail, the invention could be implemented in the health insurance entity environment according to the example as follows:

1. A health insurance entity sends an email to a diabetic patient offering a $100 annual refund on premiums at the end of the year to participate in the wellness program, along with other awards for health improvement. The patient must upload, for example, at least one month of data six times per year to qualify. In addition, the patient must collect a minimum number of glucose readings depending on his/her diabetic condition and treatment (for instance, diabetes patients who perform variable meal boluses would be asked to upload more glucose data than someone who only had daily basal injections). There would also be required measurement timing requirements, such as basal only patients would need to have at least seven waking glucose readings per time period. A URL is provided to enable the patient to sign up for the incentive program and to upload data at the specified intervals to the insurers data management system.

2. The insurer's data management system processes the data, preferably automatically upon its receipt, and sends a report to the patient. The report may include, for example, a phone number for a health advisor that the patient to contact to discuss the report and recommendations. After the first upload, the reports could identify:
a. Receipt of glucose data indicative of poor diabetes management (e.g., medically significant fluctuations in measured glucose correctable by changes in carbohydrate consumption) and instructions about desirable changes to the patient's health management regimen.
b. A current assessment of the patient's condition, along with recommendations and an incentive goal for the patient, such as an additional $100 rebate of premiums if the patient can lower his/her mean glucose by 20 mg/dL for patients participating in a continuous glucose management protocol, or discounts on test strip prices or co-pays for other patients.

3. Subsequent uploads could result in reports sent to patients with comparisons between the current and prior assessments, and with an update concerning rewards earned and redeemed. The insurer or other analyst could periodically run the patient data through an analysis program that would calculate metrics, for instance, average glucose and glucose variability over some time period(s). The analysis program would compare these metric(s) to agreed-upon predetermined thresholds.

Such reports can also be utilized to provide benefits to a third party, such as the patient's employer. Through a negotiated agreement between an insurer and the employer, improvements in pre-determined metrics for a patient or patient population could result in group insurance cost savings or other business-related incentives and rewards for the employer.

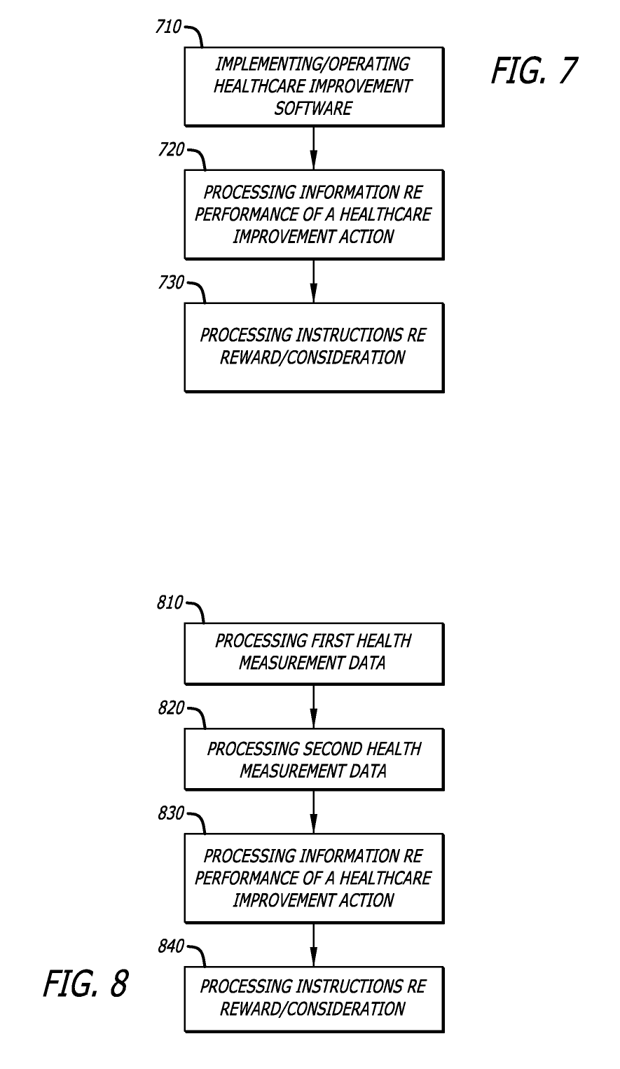

FIG. 7 is a flow chart illustrating additional health care provider environment functionality consistent with certain aspects related to the innovations herein. An exemplary method of encouraging management or improvement of patient health comprises implementing or operating health care improvement software 710 (e.g., installing or operating the Copilot™ system or accessing a health insurance entity website, etc.), processing information regarding performance of a health care improvement action or a wellness-enhancing activity 720 (e.g., processing instructions regarding providing an improved health measurement, maintaining a favorable health measurement or profile, etc.), and processing instructions or information regarding reward/consideration 730 (e.g., processing instructions regarding any of the various reward and/or consideration features herein).

FIG. 8 is a flow chart illustrating still further exemplary health care provider environment functionality consistent with certain aspects related to the innovations herein. As shown in FIG. 8, an exemplary method of encouraging management or improvement of patient health may comprise processing first health measurement data 810 (e.g., data related to original information flow 410, 420 (FIG. 4) from or related to the health care recipients, etc.), processing second health measurement data 820, processing information regarding performance of a health care improvement action or wellness-enhancing activity 830 (e.g., processing instructions regarding providing an improved health measurement, maintaining a favorable health measurement or profile, etc.), and processing instructions or information regarding reward/consideration 840 (e.g., processing instructions regarding any of the various reward and/or consideration features herein). For example, one specific implementation, here, may comprise processing several installments of patient glucose measurement data by the health care provider via a web portal 480 (FIG. 4), processing information to confirm that the patient has improved his/her glucose profile, and notifying the patient via his/her diabetes management device (e.g., CGM meter, etc.) that his/her health insurance premium is being reduced, that other rewards or incentives are available, or providing recommendations for improvements in the patient's health management regimen.

In a specific example of this embodiment, reports generated can also provide direct feedback to the patient regarding the cost of his/her treatment. A health management device (like the handheld for a continuous glucose monitoring (CGM) system or an insulin pump) or a data management system is, according to this embodiment, provided with the ability to display the report with fields for a health management improvement recommendation that correlates its performance to potential health-related expenditures saved. For instance, on a CGM handheld, the device may have an algorithm that automatically produces an exercise recommendation based on entered data, or the recommendation could be pushed to the device by an analyst as otherwise described herein. With the recommendation, an estimate is provided of the amount of money that could be saved on insulin purchases as a function of different degrees and durations of exercise.

As an example, if a patient reports having performed the exercises twice a week for twenty minutes, the report could provide an estimate to the patient that if he/she did the same exercise three times a week for thirty minutes he/she could save $23 per month in insulin costs. And if he/she exercised five times a week for twenty minutes, he/she could save $32 per month in insulin costs. Also, it could report estimates that ten fewer calories per meal would save $20 per month in insulin costs (plus the cost of food) and improve the patient's hemoglobin A1c by an estimated 0.2. Such functionality could also be provided in an exercise machine to be used by the patient; e.g., on a treadmill display, by providing the machine with software containing the requisite algorithm and a patient profile, or by connecting the machine to a health management system to which the patient has uploaded health data as described herein.

In the present description, the terms component, module, device, etc. may refer to any type of logical or functional process or blocks that may be implemented in a variety of ways. For example, the functions of various blocks can be combined with one another into any other number of modules. Each module can be implemented as a software program stored on a tangible memory (e.g., random access memory, read only memory, CD-ROM memory, hard disk drive) to be read by a central processing unit to implement the functions of the innovations herein. Or, the modules can comprise programming instructions transmitted to a general purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, implementations and features of the invention may be implemented through computer-hardware, software and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe components such as software, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various processes and operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, and so on).

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Further implementations and/or variations may be provided in addition to those set forth herein. For example, the present invention may be directed to various combinations and sub-combinations of the features disclosed in the detailed description of preferred embodiments.

While the system and method have been described in terms of what are presently considered to be specific embodiments, they need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

Other embodiments of the invention may become apparent to those skilled in the art from consideration of the specification and drawings disclosed herein. However, it is intended that the specification and drawings provided here be considered as exemplary only, with a true scope and spirit of the invention being indicated by the claims appended hereto.

What is claimed is:

1. A medical data analysis system to encourage more rigorous management of a health condition, the system comprising:
a medical sensor configured to sense a physiological parameter relevant to a particular disease-afflicted health condition and to provide medical data representative of the sensed parameter;
a processor programmed to receive the medical data that is representative of the sensed parameter and relevant to the afflicted health condition, process the received medical data, process an analysis of the afflicted health condition that is based on the medical data, and automatically determine the status of the afflicted health condition based on the analysis; and
the processor further programmed to automatically provide a level of encouragement to more rigorously manage the afflicted health condition, wherein the level of encouragement is based on the determined status of the afflicted health condition.

2. The medical data analysis system of claim 1, wherein the encouragement comprises a reward and wherein the processor is further programmed to automatically provide the level of reward based on the determined status of the afflicted health condition.

3. The medical data analysis system of claim 2, wherein the reward comprises an automatically granted discount on a health insurance cost.

4. The medical data analysis system of claim 2, wherein the reward comprises an automatically granted discount on co-payment for office visits.

5. The medical data analysis system of claim 1, wherein the encouragement comprises a penalty and wherein the processor is further programmed to provide the level of penalty based on the determined status of the afflicted health condition.

6. The medical data analysis system of claim 1, wherein the encouragement comprises a reward and wherein the processor is further programmed to a provide a reward for the more frequent sensing of a physiological parameter relevant to the particular disease-afflicted health condition and for more frequently providing medical data representative of the sensed parameter to the processor for processing.

7. The medical data analysis system of claim 1, wherein the processor is further programmed to provide a report of the processed medical data, the analysis, and the determined status of the afflicted health condition.

8. The medical data analysis system of claim 1, further comprising a memory located remotely from the processor, and wherein the processor comprises a plurality of processors, one of which is programmed to receive the medical data that is representative of the sensed parameter and relevant to the afflicted health condition, process the received medical data, and store the processed medical data in the memory.

9. The medical data analysis system of claim 8, wherein another of the plurality of processors is programmed to retrieve the stored medical data from the memory, process an analysis of the afflicted health condition that is based on the medical data, automatically determine the status of the afflicted health condition based on the analysis; and automatically provide a level of encouragement to more rigorously manage the afflicted health condition, wherein the level of encouragement is based on the determined status of the afflicted health condition.

10. The medical data analysis system of claim 8, wherein one of the plurality of processors is programmed to automatically determine the status of the afflicted health condition based on the analysis; and automatically provide a level of encouragement comprising a reward when the determined status indicates that the afflicted health condition has improved.

11. The medical data analysis system of claim 2, wherein the reward comprises at least one of an automatically granted: discount on a health insurance premium, free medical service, discounted medical service, discounted medical supplies, discounted medical equipment, coupon redeemable for medical supplies, recognition, and congratulations.

12. The medical data analysis system of claim 1, wherein the processor is further programmed to automatically vary the provided level of encouragement as a function of a change in diet.

13. The medical data analysis system of claim 1, wherein the processor is further programmed to automatically vary the provided level of encouragement as a function of the frequency of sensing the physiological parameter relevant to the particular disease-afflicted health condition and providing the sensed medical data to the processor.

14. The medical data analysis system of claim 1, wherein the processor is further programmed to store the medical data in a memory that is accessible by a health care provider and to notify the health care provider that analysis of the stored medical data is reimbursable.

15. A medical data analysis system in which the medical data is related to diabetes, the system to encourage more rigorous management of diabetes, the system comprising:
a glucose sensor configured to sense a glucose level and to provide glucose level signals;
a processor programmed to receive the glucose level signals, process the received glucose level signals, process an analysis of the diabetes based on the glucose level signals, and automatically determine the status of the diabetes based on the analysis;
wherein the analysis of the diabetes based on the glucose level signals is prepared by a health care provider;
the processor further programmed to automatically provide a level of consideration to more rigorously manage the diabetes based on the determined status of the diabetes, with consideration automatically provided as a function of at least one of: an improvement in diet, performing a wellness-enhancing activity, an improvement in glucose level measurements; and
wherein the consideration comprises at least one of: a discount on a health insurance premium, a free medical service, a discounted medical service, discounted medical supplies, discounted medical equipment, a coupon redeemable for medical supplies, recognition, and congratulations.

16. A method of encouraging improved management of a person's health, the method comprising:
sensing a physiological parameter relevant to a particular disease-afflicted health condition and providing medical data representative of the sensed parameter;
receiving and processing information regarding performance of a wellness-enhancing activity;
processing the medical data and the information regarding the wellness-enhancing activity;
analyzing the medical data representative of the sensed parameter and the information regarding the wellness-enhancing activity and providing an analysis of the status of the afflicted health condition; and
automatically providing a level of encouragement to more rigorously manage the afflicted health condition based on the analysis.

17. The method of claim 16, wherein the step of automatically providing a level of encouragement comprises automatically rewarding by providing at least one of: a discount on health insurance cost, free or discounted services, discounts on supplies, discounts on equipment, recognition, and congratulations.

18. The method of claim 16, wherein the step of automatically providing a level of encouragement comprises automatically rewarding for at least one of: an increase in frequency of measuring health-related parameters for obtaining the health data, an improvement in diet, and an improvement in the health data.

19. The method of claim 16, wherein the analysis of the status of the afflicted health condition is further automatically provided to an insurance provider or insurance billing entity to change the insurance policy cost.
